(19) United States
(12) Patent Application Publication
(10) Pub. No.: US 2012/0077263 A1
(43) Pub. Date: Mar. 29, 2012

(54) METHODS AND MATERIALS FOR ISOLATING EXOSOMES

(75) Inventors: Christopher J. Ward, Rochester, MN (US); Marina Ramirez-Alvarado, Rochester, MN (US); Marie C. Hogan, Rochester, MN (US)

(73) Assignee: MAYO FOUNDATION FOR MEDICAL EDUCATION AND RESEARCH, Rochester, MN (US)

(21) Appl. No.: 13/376,340

(22) PCT Filed: Jun. 4, 2010
(86) PCT No.: PCT/US2010/037467

§ 371 Date: Dec. 13, 2011

Related U.S. Application Data
(60) Provisional application No. 61/184,663, filed on Jun. 5, 2009.

(51) Int. Cl. C12N 5/071

(52) U.S. Cl. 435/317.1

(57) ABSTRACT

This document relates to methods and materials involved in obtaining exosomes. For example, methods and materials for obtaining exosomes from biological samples such as urine samples are provided.

### CROSS-REFERENCE TO RELATED APPLICATIONS

This application claims the benefit of priority to U.S. Provisional Application Ser. No. 61/184,663, filed Jun. 5, 2009. The disclosure of the prior application is considered part of (and is incorporated by reference in) the disclosure of this application.

### BACKGROUND

1. Technical Field

This document relates to methods and materials involved in obtaining exosomes. For example, this document relates to methods and materials for obtaining exosomes from urine samples.

2. Background Information

Exosomes are small extracellular vesicles (about 40-100 nm in diameter) that originate from renal epithelial cells including glomerular podocytes, renal tubule cells, and the cells lining the urinary draining system (Pisitkun et al., Proc. Natl. Acad. Sci. USA, 101:13368-73 (2004)). Exosomes are formed as part of the multivesicular body (MVB) pathway in which intraluminal vesicles (ILVs) progressively accumulate during endosome maturation. They are formed by inward budding and scission of vesicles from the limiting endosomal membranes (Vella et al., Eur. Biophys. J., 37:323-32 (2008)). Exosomes are released from the MVB lumen into the extracellular environment during exocytosis. During this process, certain cytosolic proteins are incorporated into the invaginating membrane, engulfed in these vesicles, thereby maintaining the same topological orientation as the plasma membrane.

Exosome functionality seems to be determined by cell-type specific polypeptides. The presence of exosomes in serum and other body fluids such as malignant effusions, urine, and bronchoalveolar lavage suggests their involvement in physiological and pathological processes (Simpson et al., Proteomics, 8:4083-99 (2008)). Their ability to bind target cells indicates that they may be capable of modulating selected cellular activities. Exosomes are thought to be involved with the removal of unwanted proteins and transfer of pathogens (i.e., HIV) between cells.

### SUMMARY

This document relates to methods and materials involved in obtaining exosomes. For example, this document relates to methods and materials for obtaining exosomes from biological samples such as urine samples. The methods and materials described herein can be used, for example, to obtain enriched samples of exosomes from biological samples (e.g., urine samples). These enriched samples can be used to determine the presence or absence of particular types of exosomes or to determine the amount of particular types of exosomes present within a mammal (e.g., a human). The presence or amount of particular types of polypeptides within an exosome sample (e.g., a urine exosome sample) can indicate that the mammal has a particular disease or disorder as described elsewhere (Zhou et al., Kidney Int., 70:1847-57 (2006); Hogan et al., J. Am. Soc. Nephrol., 20(2):278-88 (2009); and Gonzales et al., J. Am. Soc. Nephrol., 20:363-79 (2009)).

As described herein, lectins can be used to obtain exosomes from biological samples. For example, a potato (Solanum tuberosum) lectin or a Maackia amurensis II lectin can be used to obtain polycystic kidney disease exosome-like vesicles (PKD-ELVs). PKD-ELVs are exosome-like vesicles that contain one or more polypeptides (e.g., polycystin-1 (PC1), polycystin-2 (PC2), or fibrocystin/polyductin (FCP)) from the polycystic kidney disease gene. The terms “exosome” and “exosome-like vesicle” can be used interchangeably herein and in the context of urinary exosomes or urinary exosome-like vesicle refer to small extracellular vesicles (about 40-100 nm in diameter) that originate from renal epithelial cells.

In general, one aspect of this document features a method for obtaining exosomes from a biological sample. The method comprises, or consists essentially of, (a) contacting a biological sample with a lectin under conditions wherein an exosome present in the biological sample binds to the lectin to form an exosome-lectin complex, and (b) eluting the exosome from the exosome-lectin complex to obtain a sample containing the exosome, wherein the purity of exosomes present in the sample is greater than the purity of exosomes present in the biological sample. The biological sample can be a urine sample. The biological sample can be a urine sample that was centrifuged to remove cells or debris. The biological sample can be a urine sample that was dialyzed. The lectin can be a potato lectin. The lectin can be a maackia amurensis II lectin. The exosome can comprise a polycystic kidney disease gene product. The polycystic kidney disease gene product can be a polycystin-1 polypeptide, a polycystin-2 polypeptide, or a fibrocystin/polyductin polypeptide. The purity of exosomes present in the sample can be at least about 25 percent greater (e.g., at least about 25, 35, 45, 55, 65, 75, 85, 95, or more present greater) than the purity of exosomes present in the biological sample. In some cases, the purity can be such that the exosomes are between about 100 and 2000 times (e.g., between 500 and 1000 times) purier than the exosomes present in the biological sample. The purity of exosomes present in the sample can be 50 percent greater than the purity of exosomes present in the biological sample. The contacting step (a) can comprise flowing the biological sample through a column comprising a resin comprising the lectin. The resin can be sepharose. The eluting step (b) can comprise contacting the exosome-lectin complex with a carbohydrate having binding affinity for the lectin under conditions wherein the exosome is removed from the exosome-lectin complex.

In another aspect, this document features a method for obtaining exosomes from a biological sample. The method comprises, or consists essentially of, (a) contacting a biological sample with a lectin under conditions wherein an exosome present in the biological sample binds to the lectin to form an exosome-lectin complex in a solution, and (b) obtaining the exosome-lectin complex from the solution to obtain a sample containing the exosome-lectin complex, wherein the purity of exosomes present in the sample is greater than the purity of exosomes present in the biological sample. The biological sample can be a urine sample. The biological sample can be a urine sample that was centrifuged to remove cells or debris. The biological sample can be a urine sample that was dialyzed. The lectin can be a potato lectin. The lectin can be a maackia amurensis II lectin. The exosome can comprise a polycystic kidney disease gene product. The polycystic kidney disease gene product can be a polycystin-1 polypeptide, a polycystin-2 polypeptide, or a flbrocystin/polyductin polypeptide. The purity of exosomes present in the sample can be at least about 25 percent greater (e.g., at least about 25, 35, 45, 55, 65, 75, 85, 95, or more present greater) than the purity of exosomes present in the biological sample. In some cases, the purity can be such that the exosomes are between about 100 and 2000 times (e.g., between 500 and 1000 times) purier than the exosomes present in the biological sample. The purity of exosomes present in the sample can be at least about 50 percent greater than the purity of exosomes present in the biological sample. The lectin can be a biotinylated lectin. The obtaining step (b) can comprise contacting the exosome-lectin complex with a magnetic support comprising streptavidin under conditions wherein the biotinylated lectin of the exosome-lectin complex binds to the streptavidin. The obtaining step (b) can comprise using a magnetic force to obtain the magnetic support, thereby obtaining the exosome-lectin complex. The magnetic support can be a magnetic bead. The method can comprise, after the step (b), removing the exosome from the exosome-lectin complex.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a photograph of a Western blot of human urinary ELVs stained with anti-PC-1 monoclonal antibodies (7e12) (A), anti-PC-2 antibodies (polyclonal antibody to the C-terminus of a PC-2 polypeptide) (B), and monoclonal anti-FCP antibodies (C). Lanes 1, 5, and 7 contain human urinary ELVs. Lanes 2, 6, and 8 contain recombinant PC-1, PC-2, and FCP polypeptides, respectively. Lane 3 contains human urinary ELVs treated with PNGase, while lane 4 contains recombinant PC-1 polypeptide treated with PNGase.

FIG. 2 is a photograph of a Western blot of human urinary ELVs precipitated with a biotinylated lectin and streptavidin magnetic beads and stained with anti-PC-1 monoclonal antibodies (7e12). Lane 1 contains human urinary ELVs only. Lane 2 contains a no lectin precipitate control. Lane 3 contains a no lectin supernatant control. Lane 4 contains material precipitated with wheat germ agglutinin. Lane 5 contains the supernatant from the wheat germ agglutinin precipitation. Lane 6 contains material precipitated with a potato (Solanum tuberosum) lectin. Lane 7 contains the supernatant from the potato lectin precipitation. Lane 8 contains material precipitated with tomato lectin. Lane 9 contains the supernatant from the tomato lectin precipitation. The potato lectin was the most efficient at purifying PKD-ELVs.

### DETAILED DESCRIPTION

This document provides methods and materials related to isolating exosomes (e.g., exosome-like vesicles) from a biological sample. The biological sample can be any sample including, without limitation, urine, bile, plasma, saliva, and semen samples. Any appropriate volume of a biological sample can be used. For example, 5, 10, 15, 20, 25, 30, 40, 50, 75, 100, 125, 150, 175, 200, 225, 250, or more mL of urine can be used.

In some cases, the biological sample can be processed before exosomes are isolated. For example, a urine sample can be subjected to a centrifugation step to remove cells or debris. Such a centrifugation step can include spinning the sample (e.g., a urine sample) at between 12,000 to 22,000 g (e.g., about 17,000 g) for between 10 and 20 minutes (e.g., about 15 minutes). In some cases, a biological sample (e.g., a urine sample) or the resulting supernatant from a centrifugation step can be subjected to an exosome isolation process or can be subjected to a dialysis step. Such a dialysis step can include dialyzing a biological sample or the supernatant resulting from centrifugation of a biological sample against a large volume (e.g., a volume between 3 to 5 L such as about 4 L) of buffer (e.g., 100 mM MES buffer, pH 6.0) using a membrane have a molecular weight cutoff between about 5,000 Da and about 125,000 Da (e.g., between about 5,000 Da and about 100,000 Da, between about 5,000 Da and about 75,000 Da, between about 5,000 Da and about 50,000 Da, between about 10,000 Da and about 125,000 Da, between about 15,000 Da and about 125,000 Da, between about 25,000 Da and about 125,000 Da, or between about 50,000 Da and 100,000 Da). For example, a membrane having a molecular weight cutoff about 10,000 Da or about 100,000 Da can be used. This dialysis step can be repeated one, two, three, four, or more times. Once obtained, a biological sample (e.g., a centrifuged, dialyzed urine sample, a centrifuged, undialyzed urine sample, or an unprocessed urine sample) can be subjected to an exosome isolation step.

In some cases, an exosome isolation step can include using chromatography. For example, a biological sample can be allowed to flow through a gravity fed column of resin coupled with a lectin (e.g., a column with about 2 mL of Sepharose 2B cyanogen bromide coupled with a lectin). The lectin can be any lectin having the ability to bind to a carbohydrate present on an exosome. Examples of such lectins can include, without limitation, those lectins set forth in Table 1. For example, a column containing a potato lectin can be used to isolate PKD-ELVs. Once a column is loaded with a sample, the column can be washed. For example, the column can be washed one, two, three, four, five, or more times using between 2 and 10 mL of a buffer (e.g., about 5 mL of PBS). After washing the column, the isolated exosomes can be released using an excess of the natural carbohydrate recognized by the lectin. For example, 1 mL of 200 mM chitobiose can be used to elute PKD-ELVs from a column, thereby isolated PKD-ELVs.

TABLE 1
Lectins for binding to exosomes.

| Lectin | Carbohydrate | Exosome |
|---|---|---|
| Solanum tuberosum (Potato) lectin | chitobiose | PKD-ELVs |
| Maackia amurensis II lectin | salic acid | PKD-ELVs |

In some cases, an exosome isolation step can include using magnetic particles (e.g., beads) coupled to lectins. For example, a biological sample can be incubated with magnetic particles coupled to lectins such that the lectins bind to a carbohydrate present on the exosome. The lectin can be any lectin having the ability to bind to a carbohydrate present on an exosome. Examples of such lectins can include, without limitation, those lectins set forth in Table 1. For example, a magnetic bead containing a potato lectin can be used to isolate PKD-ELVs. Any appropriate method can be used to couple a lectin to a magnetic particle. For example, biotinylated lectins can be attached to streptavidin magnetic particles via the interaction between biotin and streptavidin. Once the magnetic beads containing a lectin bind the exosomes, the beads can be washed, and the exosomes released using an excess of the natural carbohydrate recognized by the lectin. For example, 1 mL of 200 mM chitobiose can be used to release PKD-ELVs from magnetic beads coupled to potato lectins, thereby isolating PKD-ELVs.

The invention will be further described in the following examples, which do not limit the scope of the invention described in the claims.

### EXAMPLES

### Example 1—Crude Preparation of Urinary ELVs

Urinary ELVs were isolated using a modified method similar to those described elsewhere (Pisitkun et al., Proc. Natl. Acad. Sci. USA, 101:13368-73 (2004) and Zhou et al., Kidney Int., 70:1847-57 (2006)). Briefly, the first void of the day was collected, and one tablet of complete proteinase inhibitor cocktail (Hoffmann-La Roche Inc., Nutley, N.J.) was added. The urine was chilled and centrifuged at 15,000×g for 15 minutes in an SLC-6000 rotor to remove cellular debris, filtered through an 8-μm nylon filter, and then centrifuged at 150,000×g in a Sorvall T-647.5 rotor for 1 hour. The pellet was resuspended in 1 ml, of PBS 1x Complete at concentration of 2 mg/mL protein. This material was mainly composed of THP and some ELVs and was referred to as crude ELVs.

To determine which PKD polypeptides are present in urinary ELVs and their relative size with respect to the recombinant polypeptides, the crude ELV preparation was analyzed via a Western blot analysis. The Western blot analysis compared the crude ELV preparation with exogenously expressed full-length polypeptides in PEAK cells (human embryo kidney cells) using antibodies to the LRR region of the PC-1 polypeptide (7e12), the N-terminus of the FCP polypeptide, and to the C-terminus of the PC-2 polypeptide.

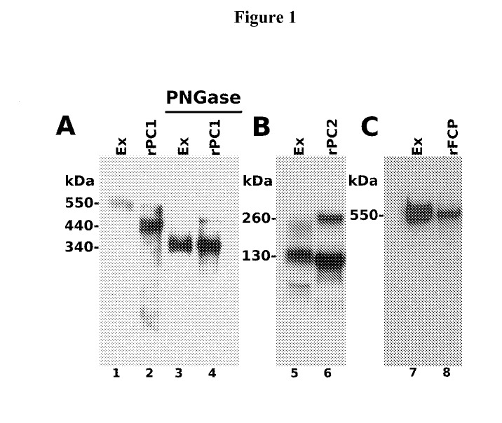

Strong signals were detected using just 2 μg of total ELY protein (compared with 10 to 50 μg of kidney membrane preparation used in previous studies of renal cells to detect PC-1 polypeptides), with the product sizes consistent with the predicted and recombinant glycosylated molecular weight of two of the polypeptides: PC-2 polypeptide (130 kD); and FCP polypeptide (500 to 550 kD) (FIGS. 1, B and C). However, the PC-1 polypeptide in ELVs was appreciably larger than the recombinant PC-1 polypeptide (FIG. 1A). To confirm that PC-1 polypeptides were specifically detected, both the ELV and the recombinant PC-1 polypeptide samples were deglycosylated, In this case, both the recombinant and ELY PC-1 polypeptides co-migrated at approximately 340 kD (predicted 325 kD), confirming identity and showing that ELY PC-1 has extensive N-linked glycosytation.

### Example 2—Isolation of Exosomes Using Chromatography

200 mL of human urine was collected and centrifuged at 17,000 g for 15 minutes to remove cells and debris. The urine was dialyzed against 4 L of 100 mM MES pH 6.0, three times using a Spectra/Por Biotech cellulose ester MWCO: 100,000 Da membrane.

The dialyzed urine sample was allowed to flow through a gravity fed column containing 2 mL of Sepharose 2B cyanogen bromide coupled with either wheat germ agglutinin, a potato lectin, a tomato lectin, or a maackia amurensis II lectin. The matrix was washed three times with 5 mL of PBS, and the column eluted with one 1 mL of 200 mM of the cognate sugar. 500 mM of N-acetylglucosamine was used in the case of wheat germ agglutinin. Chitobiose was used in the case of the potato lectin. Chitobiose was used in the case of the tomato lectin. Salic acid was used in the case of the maackia amurensis II lectin.

The collected elute was analyzed by Western blot analysis by ultra-centrifuging at 200,000 g for 1 hour and collecting the pellet in 0.25 M sucrose 20mM Tris pH 7.5.

### Example 3—Isolation of Exosomes Using Chromatography

200 mL of human urine was collected and centrifuged at 17,000 g for 15 minutes to remove cells and debris. The undialyzed urine sample was allowed to flow through a gravity fed column containing 2 mL of Sepharose 2B cyanogen bromide coupled with either wheat germ agglutinin, a potato lectin, a tomato lectin, or a maackia amurensis II lectin. The matrix was washed three times with 5 mL of PBS, and the column eluted with one 1 mL of 200 mM of the cognate sugar. 500 mM N-acetylglucosamine was used in the case of wheat germ agglutinin. Chitobiose was used in the case of the potato lectin. Chitobiose was used in the case of the tomato lectin. Salic acid was used in the case of the maackia amurensis II lectin.

The collected elute was analyzed by Western blot analysis by ultra-centrifuging at 200,000 g for 1 hour and collecting the pellet in 0.25 M sucrose 20 mM Tris pH 7.5.

### Example 4—Isolation of Exosomes Using Magnetic Beads

200 mL of human urine (centrifuged at 17,000 g for 15 minutes to remove cells) was mixed with either biotinylated wheat germ agglutinin, biotinylated potato lectin, or biotinylated tomato lectin. 100 μL of magnetic beads containing streptavidin was added to each sample. Magnetic beads were recovered by collection on a magnet, and the beads were desposited on the wall of the column. The remaining supernatant also was collected for analysis. In this case, the beads were resuspended into LiDS (Novex) for loading onto a SDS PAGE.

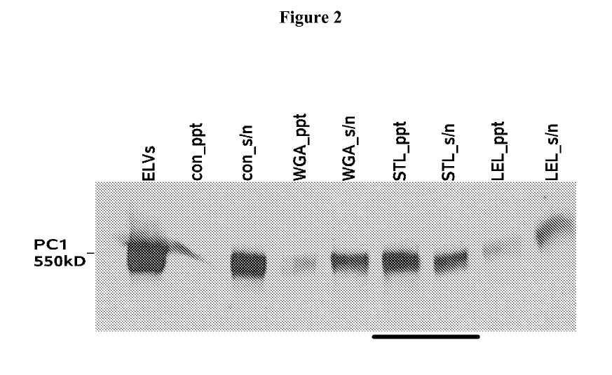

The precipitates and supernatants were analyzed by Western blot analysis using anti-PC-1 polypeptide antibodies. The positive control was ELVs obtained by ultracentrifuging urine at 200,000 g for 2 hours. No lectin controls (precipitate and supernatant) revealed the presence of PC-1 positive exosomes in the supernatant as opposed to the precipitate (FIG. 2; lanes 2 and 3). The potato lectin was the most efficient at purifying PKD-ELVs (FIG. 2).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for obtaining exosomes from a biological sample, wherein said method comprises:
(a) contacting a biological sample with a lectin under conditions wherein an exosome present in said biological sample binds to said lectin to form an exosome-lectin complex, and
(b) eluting said exosome from said exosome-lectin complex to obtain a sample containing said exosome,
wherein the purity of exosomes present in said sample is greater than the purity of exosomes present in said biological sample.

2. The method of claim 1, wherein said biological sample is a urine sample.

3. The method of claim 1, wherein said biological sample is a urine sample that was centrifuged to remove cells or debris.

4. The method of claim 1, wherein said biological sample is a urine sample that was dialyzed.

5. The method of claim 1, wherein said lectin is a potato lectin.

6. The method of claim 1, wherein said lectin is a maackia amurensis II lectin.

7. The method of claim 1, wherein said exosome comprises a polycystic kidney disease gene product.

8. The method of claim 7, wherein said polycystic kidney disease gene product is a polycystin-1 polypeptide, a polycystin-2 polypeptide, or a fibrocystin/polyductin polypeptide.

9. The method of claim 1, wherein said purity of exosomes present in said sample is 25 percent greater than the purity of exosomes present in said biological sample.

10. The method of claim 1, wherein said purity of exosomes present in said sample is 50 percent greater than the purity of exosomes present in said biological sample.

11. The method of claim 1, wherein said contacting step (a) comprises flowing said biological sample through a column comprising a resin comprising said lectin.

12. The method of claim 11, wherein said resin is sepharose.

13. The method of claim 11, wherein said eluting step (b) comprises contacting said exosome-lectin complex with a carbohydrate having binding affinity for said lectin under conditions wherein said exosome is removed from said exosome-lectin complex.

14. A method for obtaining exosomes from a biological sample, wherein said method comprises:
(a) contacting a biological sample with a lectin under conditions wherein an exosome present in said biological sample binds to said lectin to form an exosome-lectin complex in a solution, and
(b) obtaining said exosome-lectin complex from said solution to obtain a sample containing said exosome-lectin complex,
wherein the purity of exosomes present in said sample is greater than the purity of exosomes present in said biological sample.

15. The method of claim 14, wherein said biological sample is a urine sample.

16. The method of claim 14, wherein said biological sample is a urine sample that was centrifuged to remove cells or debris.

17. The method of claim 14, wherein said biological sample is a urine sample that was dialyzed.

18. The method of claim 14, wherein said lectin is a potato lectin.

19. The method of claim 14, wherein said lectin is a maackia amurensis II lectin.

20. The method of claim 14, wherein said exosome comprises a polycystic kidney disease gene product.

21. The method of claim 20, wherein said polycystic kidney disease gene product is a polycystin-1 polypeptide, a polycystin-2 polypeptide, or a fibrocystin/polyductin polypeptide.

22. The method of claim 14, wherein said purity of exosomes present in said sample is 25 percent greater than the purity of exosomes present in said biological sample.

23. The method of claim 14, wherein said purity of exosomes present in said sample is 50 percent greater than the purity of exosomes present in said biological sample.

24. The method of claim 14, wherein said lectin is a biotinylated lectin.

25. The method of claim 24, wherein said obtaining step (b) comprises contacting said exosome-lectin complex with a magnetic support comprising streptavidin under conditions wherein the biotinylated lectin of said exosome-lectin complex binds to said streptavidin.

26. The method of claim 25, wherein said obtaining step (b) comprises using a magnetic force to obtain said magnetic support, thereby obtaining said exosome-lectin complex.

27. The method of claim 25, wherein said magnetic support is a magnetic bead.

28. The method of claim 14, wherein said method comprising, after said step (b), removing said exosome from said exosome-lectin complex.
